(19) United States
(12) Patent Application Publication
(10) Pub. No.: US 2011/0251643 A1
(43) Pub. Date: Oct. 13, 2011

(54) Spinal Osteosynthesis System

(76) Inventor: Lotfi Miladi, Bourg La Reine (FR)

(21) Appl. No.: 13/125,340

(22) PCT Filed: Oct. 23, 2009
(86) PCT No.: PCT/FR2009/001246

§ 371 Date: Jun. 29, 2011

(30) Foreign Application Priority Data
Oct. 23, 2008 (FR) 08/05881

(51) Int. Cl. A61B 17/70

(52) U.S. Cl. 606/254; 606/264

(57) ABSTRACT

A spinal osteosynthesis system comprising at least one flexible rod mounted to slide freely and guided in pivoting in a member (12) for connection to means for fastening on a vertebra, such as a pedicular screw (10) for example. The invention makes it possible to strengthen and stabilize the column while preserving its mobility and without preventing growth in a child.

The invention relates to a spinal osteosynthesis system.

Traditional spinal osteosynthesis systems are designed to perform arthrodesis of the spinal column and they generally comprise two parallel rigid rods that are fastened by hooks or by pedicular screws to the vertebrae that are to be united, together with transverse connection elements between the two rods, the rods being held stationary on the hooks and the pedicular screws, in general by plugs or other locking means.

Those systems present a certain number of drawbacks:
they form assemblies that are rigid and not deformable, thereby stiffening the back and preventing any bending of the vertebral column;
when they are implanted in children, they oppose growth of the vertebral column; and
they give rise to long-term anatomic and functional damage to zones adjacent to the spinal column and that are left free, because those zones are then subjected to excess mobility, which leads to arthrosis, pain, etc.

Osteosynthesis systems have already been proposed for limiting some of those drawbacks, at least in part; systems in which the rigid rods are slidably mounted in supports (e.g. US 2006/0241594-A1), those supports possibly themselves being mounted pivotally on pedicular screws (e.g. EP 1 665 944-A1), or in which the rigid rods are replaced by a plurality of elements that are articulated one to another and that are associated with clamping means enabling them to be locked in a given orientation or position (e.g. FR 2 715 825-A1).

Nevertheless, those systems have their own drawbacks:
the system described in EP 1 665 994-A1 is made up of a large number of different parts and it is relatively heavy and bulky; and
the system described in FR 2 715 825-A1 is rigid and not deformable once implanted and it opposes bending of the spinal column and it also opposes growth thereof.

It is also known (WO 2004/010881) to fasten rigid rods on vertebrae, not by means of pedicular screws, but by means of flexible ties that connect a rod support part to a rib or to a transverse process.

A particular object of the present invention is to mitigate the above-mentioned drawbacks of prior art systems and to avoid arthrodesis of the spinal column by means of a spinal osteosynthesis system that is simpler, lighter, and less bulky than those of the prior art, and that does not oppose the mobility or the flexibility of the spinal column, and that does not oppose growth in a child.

To this end, the invention provides a spinal osteosynthesis system comprising at least one longitudinal rod connected to fastener means such as hooks or pedicular screws, for fastening to vertebrae, the system being characterized in that the rod is flexible and slides freely in its operating position relative to at least some of the fastener means.

The flexibility of the rod and its ability to slide relative to the means for fastening to the vertebrae enable the system to straighten the spinal column and to deform without exerting forces on the vertebrae in order to follow bending of the back, which thus retains its mobility. This also preserves growth of the spinal column in a child without it being necessary to modify the implanted system by repeated interventions.

According to another characteristic of the invention, the flexible rod is made of a biocompatible plastics material such as polyether etherketone (PEEK).

This material is a semicrystalline thermoplastic that is biocompatible and implantable, sterilizable, and that has mechanical characteristics in terms of strength and breaking that are similar to those of metals, and that conserves those characteristics after being implanted.

The flexibility of that rod, which is much greater than the flexibility of a metal rod having the same dimensions, contributes to preserving the mobility of the back of a patient having the system of the invention implanted thereon.

According to other characteristics of the invention, the rod is guided to move in translation in connection members for connecting to the fastener means for fastening on the vertebrae, and each connection member comprises an annular body through which the rod passes and means for mounting said body on an above-mentioned fastener means.

Advantageously, each connection member is mounted by resilient snap-fastening on an above-mentioned fastener means and includes two lateral tabs that are terminated by means for snap-fastening in complementary means of the fastener means.

This characteristic makes it much easier to implant the system, since implanting then consists in mounting all of the connection members needed on the flexible rod prior to inserting the rod and subsequently fastening said connection members by snap-fastening e.g. onto pedicular screws that have already been fastened on the vertebrae, which would not be possible if the rod were rigid.

In the operating position (in the implanted state), the rod can pivot in at least some of the connection members about a transverse axis that is perpendicular to the axis of the rod.

In addition, in each connection member, the rod is preferably prevented from pivoting about an axis that is perpendicular to the rod and to the plane of the spinal column, i.e., and for example, about the axis of a pedicular screw that constitutes fastener means for fastening to a vertebra.

The invention also provides locking means for preventing the rod from moving in translation in at least one of the above-mentioned connection means.

The rod can thus be locked, preventing it from moving in translation at one end or between its ends relative to one or more of the means for fastening to the vertebrae, in particular in order to maintain a predetermined distance between selected vertebrae.

According to another characteristic of the invention, the system comprises two parallel flexible rods connected to means for fastening to the vertebrae and united by transverse connections.

These transverse connections are rigid and fastened to some of the above-mentioned connection members by means of small screws.

As explained above, the fastener means comprise pedicular screws or hooks, the hooks advantageously comprising clamping collars made of a flexible and biocompatible material such as a plastics material of the PEEK type, for example, the clamping collars enabling fastening to be performed on the vertebrae via the laminae or transverse processes or on the ribs in the chest region.

The invention can be better understood and other characteristics, details, and advantages thereof appear more clearly on reading the following description made by way of example with reference to the accompanying drawings, in which:

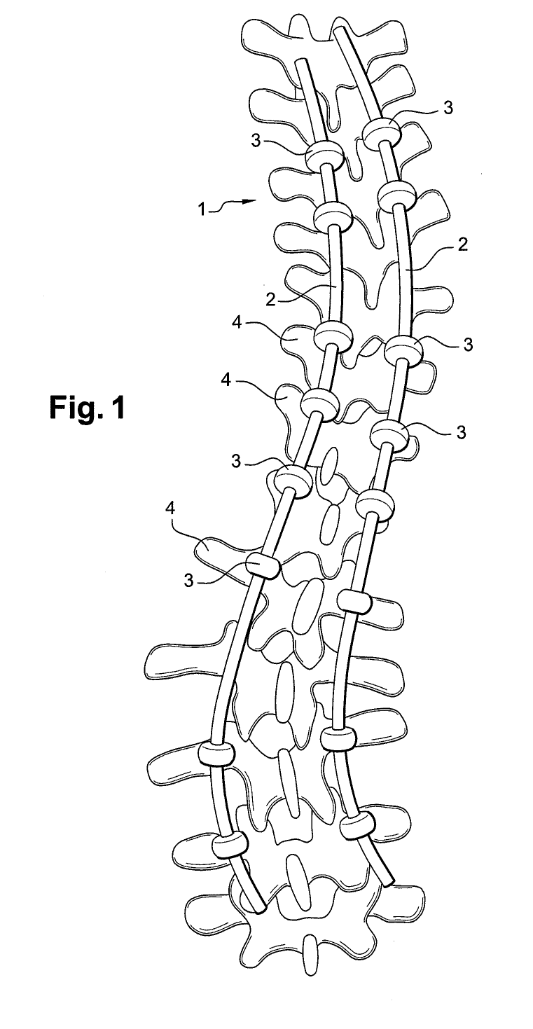

FIG. 1 is a diagrammatic view of a vertebral column including an osteosynthesis system of the invention;

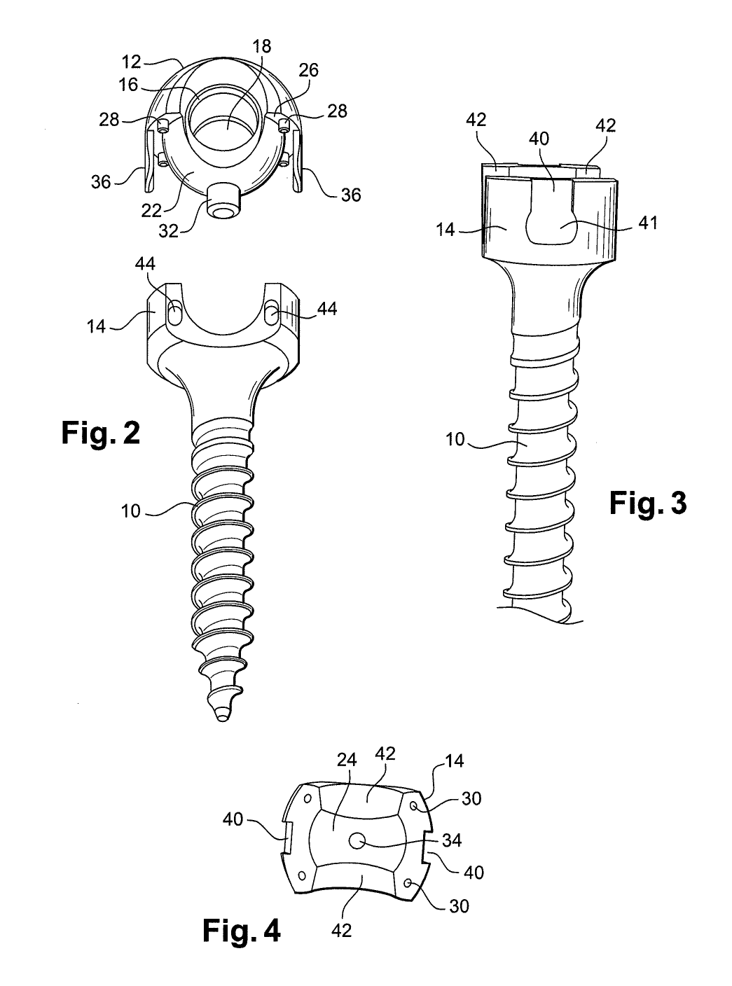

FIG. 2 is an exploded diagrammatic view in perspective of pedicular fastener means of the invention;

FIG. 3 is a diagrammatic view in perspective of the FIG. 2 screw;

FIG. 4 is a plan view of a screw socket;

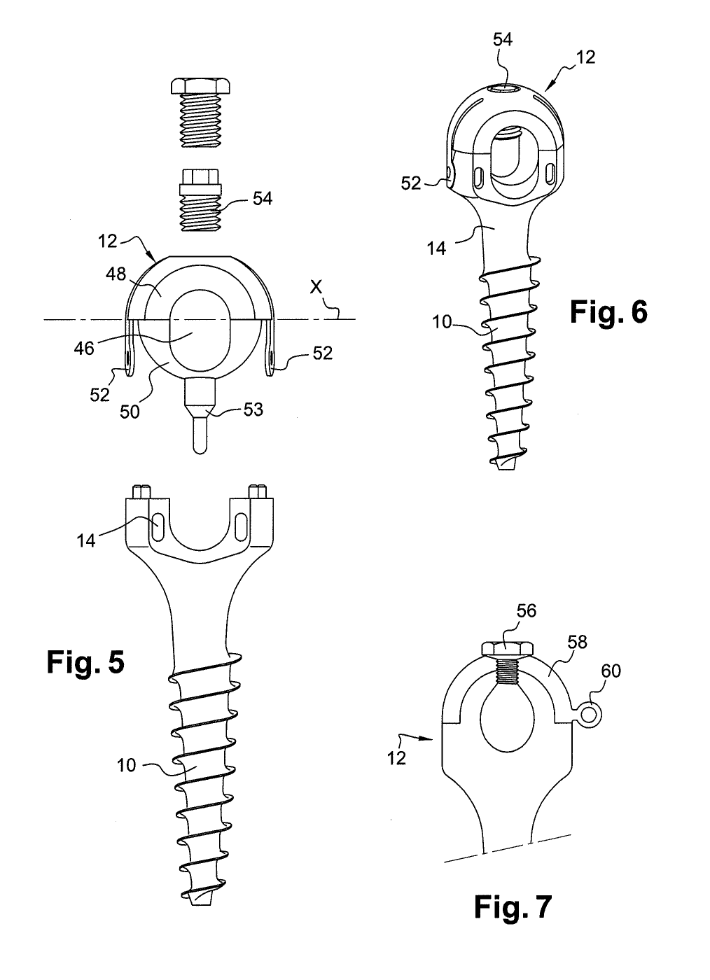

FIG. 5 is an exploded diagrammatic view in perspective of a variant embodiment of the invention;

FIG. 6 is a diagrammatic view in perspective of the FIG. 5 device;

FIG. 7 is a diagrammatic view of fastener means for a lateral connection with other fastener means;

FIG. 8 is a perspective view of an indexing hook; and

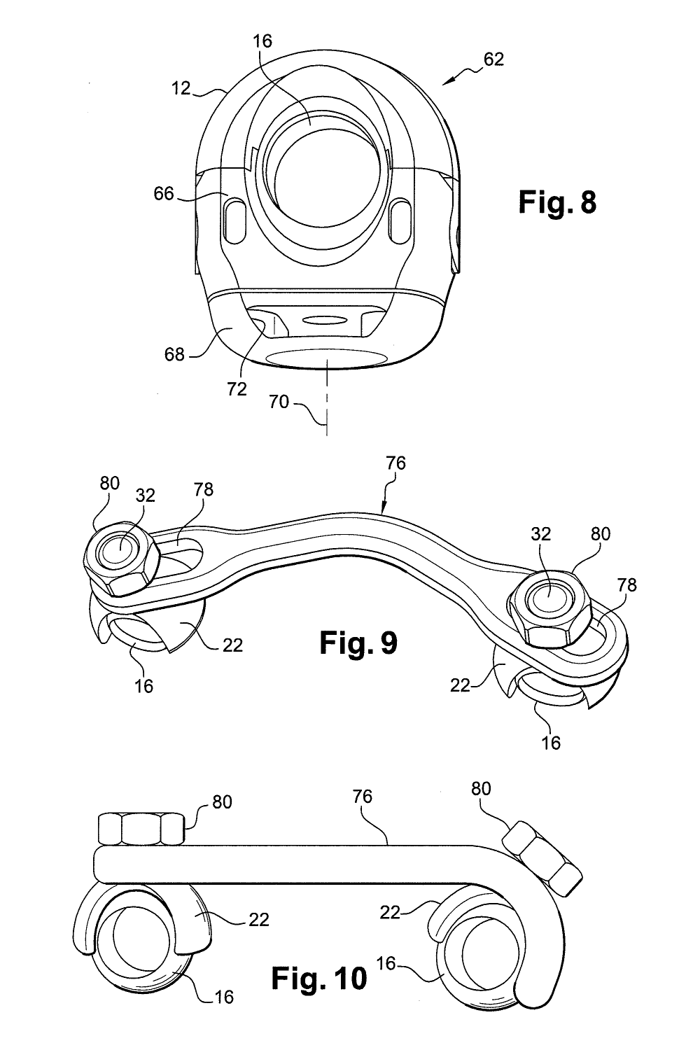

FIGS. 9 and 10 are perspective views of transverse connection devices.

FIG. 1 is a diagram showing a vertebral column 1 and an osteosynthesis system of the invention, the system essentially comprising two parallel longitudinal rods 2 guided in members 3 for connection to means for fastening on the vertebrae 4.

Each rod 2 is securely mounted in at least one of the connection members 3 and is capable of sliding and pivoting in the other connection members, thereby enabling the mobility of the patient's back to be conserved and making it possible, in a child, for the column to grow.

Each rod 2 is also flexible, thereby allowing it to follow movement of the back, while keeping the column in alignment.

FIGS. 2 to 4 show pedicular screw fastener means of the invention, said means comprising a threaded shank 10 of conventional or fluted type (enabling it to be engaged on a guide pin), made of metal (e.g. of titanium or stainless steel), and a socket 14 formed at the top end of the threaded shank 10, a connection member 12 being fastened on the socket 14 by resilient snap- or clip-fastening.

The member 12 includes a housing for receiving and guiding a ball 16 having a bore 18 passing axially therethrough and receiving a flexible cylindrical rod 2 that extends with clearance so as to be capable of sliding freely in the ball 16.

In this embodiment of the invention, the semi-annular bottom portion 22 of the member 12 forms a guide surface 24 for the ball 16, it is fitted on the top portion of the member 12, and is fastened thereto by any appropriate means, e.g. by welding or by adhesive.

The base 26 of the member 12 includes means such as pegs 28 for positioning and centering on the socket 14 of the screw 10, which means are received in small holes 30 in the top face of the socket, an additional axial peg 32 being formed on the semi-annular part 22 and being received in a central hole 34 of the surface 24 of the socket 14.

The member 12 is fastened on the socket 14 by means of two parallel tabs 36 formed at the ends of a U-shaped strap mounted in a semicircular groove of the outside surface of the member 12, the two tabs 36 being designed to engage in two lateral cavities or grooves 40 in the socket 14 and including snap-fastener means at their ends that engage resiliently in complementary means provided at the bottom ends of the grooves 40.

By way of example, the ends of the tabs 36 may present respective enlarged portions designed to be received in respective enlarged ends 41 of the lateral grooves 40 in the socket 14, as shown in FIG. 3.

Thus, it suffices to position the connection member 12 on the socket 14 by engaging the pegs 28 and 32 in the orifices 30 and 34 in the socket, and to push it against the socket so that the tabs 36 engage and become held in the grooves 40.

The member 12 may be separated from the socket 14 on splaying apart the ends of the tabs 36 while exerting traction on the member 12 so as to move it away from the base 14.

As can be seen in FIG. 4, the spherical inside surface 24 of the socket 14 lies between two frustoconical conical surfaces 42 that flare going towards the outside faces of the socket 14 so as to enable the rod 2 to move angularly relative to the screw 10.

Similarly, a spherical inside surface of the top portion of the member 12 is formed between two frustoconical surfaces corresponding to the surfaces 42 of the socket 14.

The angular movement of the rod 2 about its center of rotation in the connection member 12 may for example be of the order of 15° to 35° in all directions. The angular movement about the axis of the screw 10 is preferably restricted or substantially zero.

The flat outside faces of the socket 14 also include symmetrical setbacks or imprints 44 used for fastening a tool on the socket for the purpose of manipulating and inserting the fastener screw.

The embodiment shown in the drawings has the advantage that the ball 16 for guiding the rod 2 is held captive in the connection member 12 and cannot escape therefrom.

The flexible rod 2 is made of a biocompatible material, preferably such as a PEEK type plastics material, which may be given a suitable curved shape by molding or by thermoforming. PEEK has a bending modulus of 4 gigapascals (GPa).

The rod 2 has a diameter lying in the range 4 millimeters (mm) to 6 mm, for example, and it may have a section that is circular over its entire length or indeed that is non-circular over all or part of its length, in which case it is guided in a passage 18 of complementary shape in the ball 16 so as to allow the rod 2 to slide freely in the axial direction while preventing it from turning about its own axis, and thus opposing any twisting of the rod 2.

It is also possible to use rods 2 of different diameters and different flexibilities depending on the implantations that are to be performed.

In a variant, the ball 16 is replaced by a cylindrical guide member, that like the ball 16 includes a through bore or passage for guiding the rod 2 in translation, the axis of the cylindrical guide member extending transversely and perpendicularly to the axis of the rod. The bore presents an enlargement at each of its ends to allow the rod to pivot about an axis that is perpendicular to the axis of the screw 10 and to the axis of the rod 2, and that extends approximately between the two resilient snap-fastener tabs 36 of the connection member 12.

FIGS. 5 and 6 show another embodiment of the invention, that differs from the embodiment described above in that the connection member 12 does not have a ball 16, said member being constituted essentially by an annular body presenting a central opening 46 of oblong shape through which the flexible rod 2 passes and is free to slide in said opening along its own axis and can pivot about a transverse axis X that is perpendicular to the axis of the rod 2 and to the axis of the screw 10.

Preferably, pivoting of the rod 2 in the opening 46 about the axis of the screw 10 is zero or substantially zero. For this purpose, it suffices that the width of the opening 46 along the transverse axis X is slightly greater than the diameter of the rod 2.

The connection member 12 is made up of two semi-annular parts 48 and 50 that are fastened to each other by laser welding or by tungsten inert gas (TIG) welding, and they form an annular body that is fastened to the socket 14 of the screw 10 by resilient snap- or clip-fastening using lateral tabs 52, these tabs being fitted to the member 12 or formed integrally therewith.

In this embodiment, positioning pins of the member 12 are formed to project from the top face of the socket 14 and are received in corresponding holes in the bottom face of the member 12, and an axial finger 53 is formed to project from the bottom portion 50 of said member and to be received in a central passage of the socket 14 for the purpose of centering and guiding the member 12.

The top portion 48 of the member 12, on its side remote from the screw 10, includes a tapped orifice that extends along the axis of the screw 10 and that receives a lock screw 54 for locking the flexible rod 2 in the connection member 12, this lock screw possibly also forming means for limiting any pivoting of the rod 2 about the transverse axis X.

By means of another screw 56 engaged in the tapped orifice, it is also possible to fasten lateral connection means onto the connection member 12, as shown in FIG. 7.

These lateral connection means comprise a semi-annular part 58 fastened on the top portion 48 of the member 12 by the screw 56, this part 58 having a lateral lug 60 at one end that is formed with a central hole for attaching to one end of a lateral connection rod that passes over a plurality of vertebrae and that has its other end fastened in the same way to another connection member 12 mounted on the same flexible rod 2.

FIG. 8 shows a hook 62 for fastening on a vertebra or a rib, the hook comprising a connection member 12 identical to that described above, that is fastened by resilient snap-fastening onto a socket 66 having a bottom portion 68 mounted to pivot about a central axis 70 that is substantially vertical in the drawing. The bottom portion 68 of the socket includes a slot 72 for passing a flexible tie, e.g. made of polymer such as PEEK, acting as a clamping collar and enabling the hook 62 to be fastened onto a process of a vertebra or onto a rib of the rib cage. In particular, it is possible to fasten the hooks 62 on cervical vertebrae, thus avoiding any need to implant screws in those vertebrae that are highly mobile and run the risk of subjecting the bone to wear or lysis.

As mentioned above, the device of the invention may comprise two substantially parallel flexible rods 2 that are united from place to place by transverse connection devices, such as those shown in FIGS. 9 and 10.

Each transverse connection device comprises a straight or curved rigid bar 76 having its ends including oblong slots 78 for passing respective threaded rods, e.g. constituted by the central peg 32 of the guide part 22 of a ball 16 (FIG. 2). A nut 80 screwed onto each peg 32 serves to fasten each part 22 onto the bar 76, so as to maintain a predetermined spacing between the rods 2 that pass through the balls 16 of the parts 22.

The axes of the pegs 32 may be substantially perpendicular to the bar 76 as shown in FIG. 9 or they may be inclined relative to the bar as shown in the right-hand portion of FIG. 10 so that the nuts 80 project less under the patient's skin, in particular for a child, with the end of the bar 76 being curved or inclined for this purpose.

The transverse bars 76 may also be fastened to the connection members 12 such as that shown in FIG. 5, by means of screws engaged in the tapped orifices in the top portions 48 of the members 12.

1. A spinal osteosynthesis system comprising at least one longitudinal rod connected to fastener means for fastening to vertebrae, said rod being flexible and sliding freely in its operating position in connection members that are fastened by resilient snap-fastening to the fastener means for fastening to the vertebrae.

2. A system according to claim 1, wherein the rod is held stationary in at least one of the connection members for connecting it to fastener means for fastening to the vertebrae.

3. A system according to claim 1, wherein in the operating position, the rod can pivot, in at least some of the connection members, about a respective transverse axis perpendicular to the axis of the rod.

4. A system according to claim 3, wherein in each connection member, the rod is prevented from pivoting about an axis perpendicular to the axis of the rod and to the above-mentioned transverse axis.

5. A system according to claim 1, wherein the connection members include lateral tabs that terminate in snap-fastener means for engaging in complementary means of the fastener means.

6. A system according to claim 1, wherein each connection member includes positioning and centering means for positioning and centering it on a socket of fastener means.

7. A system according to claim 1, wherein the rod is made of a biocompatible plastics material.

8. A system according to claim 1, wherein the rod is, at least in part, of section that is not circular, and each connection member includes a passage of corresponding section through which the rod passes with clearance, thereby opposing turning of said rod about its axis.

9. A system according to claim 1, wherein some of the connection members for connecting to the fastener means for fastening on the vertebrae include screws for locking the rod.

10. A system according to claim 1, comprising two parallel longitudinal flexible rods connected to means for fastening on the vertebrae and united with each other by transverse connections fastened at their ends on connection members mounted on the flexible rods.

11. A system according to claim 1, wherein the means for fastening on the vertebrae comprise pedicular screws or hooks for clamping collars made of a flexible and biocompatible material.

12. A system according to claim 1, wherein the rod is made of polyether etherketone (PEEK).

13. A system according to claim 1, wherein the means for fastening on the vertebrae comprise pedicular screws or hooks for clamping collars made of a flexible and biocompatible material comprising PEEK.

14. A spinal osteosynthesis system comprising at least one longitudinal rod connected to one or more fasteners configured to fasten to vertebrae, said rod being flexible and sliding freely in its operating position in connection members that are fastened by resilient snap-fastening to the one or more fasteners for fastening to the vertebrae, and wherein the rod is held stationary in at least one of the connection members.
